(19) United States
(12) Patent Application Publication
(10) Pub. No.: US 2025/0347706 A1
(43) Pub. Date: Nov. 13, 2025

(54) DISPOSABLE CARTRIDGE FOR CLASSIFICATION OF ANTICOAGULANT AND METHOD OF USE

(71) Applicant: Haemonetics Corporation, Boston, MA (US)

(72) Inventors: Gerard R. DelGiacco, Yonkers, NY (US); Ebrahim Shafizadeh, North Easton, MA (US)

(21) Appl. No.: 19/279,581

(22) Filed: Jul. 24, 2025

Related U.S. Application Data
(63) Continuation of application No. PCT/US24/12894, filed on Jan. 25, 2024.
(60) Provisional application No. 63/482,775, filed on Feb. 1, 2023.
(60) Provisional application No. 63/482,982, filed on Feb. 2, 2023.

(51) Int. Cl. G01N 33/86; B01L 3/00

(52) U.S. Cl. CPC G01N 33/86; B01L 3/502715; B01L 2200/04; B01L 2200/16; B01L 2300/087

(57) ABSTRACT

Disposable cartridges and methods for detecting and classifying an anticoagulant at a therapeutically relevant amount or higher in a patient. The cartridges include a plurality of chambers, as well as fluidic couplings between chambers. Calcium chloride, Russel Viper Venom (RVV), an ecarin reagent, a Factor Xa reagent, protamine, kaolin, tissue factor, and combinations thereof may be included in a given chamber.

### CROSS-REFERENCE TO RELATED APPLICATIONS

The present application is a continuation application of International Patent Application No. PCT/US2024/012894, which was filed on Jan. 25, 2024, which claims priority from U.S. Provisional Patent Application No. 63/482,775 filed Feb. 1, 2023, and from U.S. Provisional Patent Application No. 63/482,982 filed Feb. 2, 2023 respectively, all of which are incorporated herein by reference.

### TECHNICAL FIELD

The present invention relates to devices and methods for detecting and classifying an anticoagulant at a therapeutically relevant amount or higher in a patient.

### SUMMARY OF THE EMBODIMENTS

In accordance with an embodiment of the invention, a disposable cartridge for detecting and classifying an anticoagulant at a therapeutically relevant amount or higher in a patient, the cartridge comprising (1) a first channel having (a) a first receiving chamber, comprising calcium chloride, configured to receive a first sample of a blood component from the patient, (b) a first reagent chamber, comprising Russel Viper Venom (RVV), fluidically coupled to the first receiving chamber and configured to receive the first sample from the first receiving chamber, and (c) a first measurement chamber fluidically coupled to the first reagent chamber and configured to receive the first sample from the first reagent chamber, the first measurement chamber being further configured for viscoelastic analysis of the first sample received from the first reagent chamber so as to provide a first clotting measurement of the first sample received from the first reagent chamber; and (2) a second channel having (d) a second receiving chamber configured to receive a second sample of the blood component from the patient, (e) a second reagent chamber, comprising an ecarin reagent, fluidically coupled to the second receiving chamber and configured to receive the second sample from the second receiving chamber, and (f) a second measurement chamber fluidically coupled to the second reagent chamber and configured to receive the second sample from the second reagent chamber, the second measurement chamber being further configured for viscoelastic analysis of the second sample received from the second reagent chamber so as to provide a second clotting measurement of the second sample received from the second reagent chamber. The first clotting measurement may be a first R-time. The second clotting measurement may be a second R-time.

In some embodiments, the disposable cartridge further comprises (3) a third channel having (g) a third receiving chamber configured to receive a third sample of the blood component from the patient, (h) a third reagent chamber, comprising a Factor Xa reagent and calcium chloride, fluidically coupled to the third receiving chamber and configured to receive the third sample from the third receiving chamber, and (i) a third measurement chamber fluidically coupled to the third reagent chamber and configured to receive the third sample from the third reagent chamber, the third measurement chamber being further configured for viscoelastic analysis of the third sample received from the third reagent chamber so as to provide a third clotting measurement of the third sample received from the third reagent chamber. The third clotting measurement may be a third R-time.

In some embodiments, the disposable cartridge further comprises (4) a fourth channel having (j) a fourth receiving chamber, comprising protamine, configured to receive a fourth sample of the blood component from the patient, (k) a fourth reagent chamber fluidically coupled to the fourth receiving chamber and configured to receive the fourth sample from the fourth receiving chamber, and (l) a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber. The fourth reagent chamber may comprise a reagent selected from the group consisting of kaolin, tissue factor, calcium chloride, and combinations thereof. The fourth clotting measurement may be a fourth R-time.

In other embodiments, the disposable cartridge further comprises (4) a fourth channel having (j) a fourth receiving chamber, comprising a heparinase reagent, configured to receive a fourth sample of the blood component from the patient, (k) a fourth reagent chamber, comprising kaolin, tissue factor, and calcium chloride, fluidically coupled to the fourth receiving chamber and configured to receive the fourth sample from the fourth receiving chamber, and (l) a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber.

In accordance with another embodiment of the invention, a method for detecting a presence of and classifying an anticoagulant at a therapeutically relevant amount or higher in a patient, the method comprising (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, and (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently.

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, and (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher; wherein (a) the first R-time greater than the first control R-time, the second R-time less than or equal to the second control R-time, and the third R-time greater than the third control R-time indicates the presence of the Factor Xa inhibitor at or above a therapeutically relevant amount in the patient; (b) the first R-time greater than the first control R-time, the second R-time greater than the second control R-time, and the third R-time greater than the third control R-time indicates the presence of the DTI at or above a therapeutically relevant amount in the patient; and (c) the first R-time less than or equal to the first control R-time, the second R-time less than or equal to the second control R-time, and the third R-time greater than the third control R-time indicates the presence of the vitamin K antagonist at or above a therapeutically relevant amount in the patient.

In accordance with another embodiment of the invention, a method for detecting a presence of and classifying an anticoagulant at a therapeutically relevant amount or higher in a patient, the method comprising (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and (D) determining a fourth R-time by subjecting a fourth sample of the blood component from the patient to a clotting assay in the presence of protamine.

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher, and (iv) comparing the fourth R-time to a fourth control R-time, said fourth control R-time being derived from a fourth set of control blood component samples, each control blood component sample of the fourth set having been obtained from a control patient known to lack each of a Factor Xa inhibitor, a DTI, a vitamin K antagonist, and heparin, at a therapeutically relevant amount or higher; wherein (a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient; (b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient; (c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient; (d) the first R-time greater than the first control R-time, the second R-time less than or equal to the second control R-time, the third R-time greater than the third control R-time, and the fourth R-time less than the fourth control R-time indicates the presence of heparin at or above a therapeutically relevant amount in the patient. In some embodiments, the fourth sample of the blood component from the patient may subjected to the clotting assay in the presence of the protamine and an additional reagent selected from the group consisting of kaolin, tissue factor, calcium chloride, and combinations thereof, said fourth sample of the blood component from the patient being admixed with the protamine prior to being admixed with the additional reagent.

In accordance with another embodiment of the invention, a method for detecting a presence of and classifying an anticoagulant at a therapeutically relevant amount or higher in a patient, the method comprising (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and (D) determining a fourth R-time by subjecting a fourth sample of the blood component from the patient to a clotting assay in the presence of a heparinase reagent, kaolin, tissue factor, and calcium chloride, said fourth sample of the blood component from the patient being admixed with the heparinase reagent prior to being admixed with the kaolin, tissue factor, and calcium chloride; and

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher; (iv) comparing the fourth R-time to a fourth control R-time, said fourth control R-time being derived from a fourth set of control blood component samples, each control blood component sample of the fourth set having been obtained from a control patient known to have a control anticoagulant selected from the group consisting of a Factor Xa inhibitor, a DTI, a vitamin K antagonist, and combinations thereof, at a therapeutically relevant amount or higher, and (v) comparing the second R-time to a fifth control R-time, said fifth control R-time being derived from a fifth set of control blood component samples, each control blood component sample of the fifth set having been obtained from a control patient known to lack heparin at a therapeutically relevant amount or higher; wherein (a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient; (b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient; (c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient; (d) the first R-time greater than the first control R-time, the second R-time greater than the fifth control R-time, the third R-time greater than the third control R-time, and the fourth R-time less than the fourth control R-time indicates the presence of heparin at or above a therapeutically relevant amount in the patient.

The Factor Xa inhibitor may be rivaroxaban, edoxaban, or apixaban.

The DTI may be argatroban, melagatran, ximelagatran, or dabigatran.

The vitamin K antagonist may be warfarin.

In some embodiments, the patient is administered a reversal agent so as to reduce the anticoagulant effect of the anticoagulant detected to be present at the therapeutically relevant amount or above in the patient.

### BRIEF DESCRIPTION OF THE DRAWINGS

The foregoing features of embodiments will be more readily understood by reference to the following detailed description, taken with reference to the accompanying drawings, in which:

FIG. 1 is a schematic diagram showing the clotting cascade that leads to the formation of a fibrin clot made of cross-linked fibrin.

FIG. 2 is an exemplary disposable cartridge suitable for detecting and classifying an anticoagulant at a therapeutically relevant amount or higher in a patient, in accordance with an embodiment of the invention. Fluidic couplings are not shown.

FIG. 3 is a plot showing R-time measurements conducted using a first channel (Russell's Viper Venom and calcium chloride, separated) of a cartridge described herein, in accordance with an embodiment of the invention.

FIG. 4 is a plot showing R-time measurements conducted using a second channel (ecarin) of a cartridge described herein.

FIG. 5 is a plot showing R-time measurements conducted using a third channel (Russell's Viper Venom and calcium chloride, combined) of a cartridge described herein.

### DETAILED DESCRIPTION OF SPECIFIC EMBODIMENTS

Definitions. As used in this description and the accompanying claims, the following terms shall have the meanings indicated, unless the context otherwise requires.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

A “set” includes at least one member.

“Blood component” is means one or more components of blood taken, for example, from a patient, where the blood component contains a sufficient quantity of plasma to form a fibrin mediated clot. The blood component may contain at least about 8% plasma on a volume basis (i.e., 8% v/v plasma). The blood component may contain at least about 10% v/v plasma, or at least about 12% v/v plasma, or at least about 15% v/v plasma, or at least about 20% v/v plasma. The patient may be a human, but may also be any other animal (e.g., veterinary animal or exotic animal). Blood is the circulating tissue of an organism that carries oxygen and nutritive materials to the tissues and removes carbon dioxide and various metabolic products for excretion. Blood includes a pale yellow or gray yellow fluid, plasma, in which are suspended red blood cells, white blood cells, and platelets. Blood (sometimes referred to as whole blood) can be fractionated into various components or fractions following density gradient centrifugation. Thus, a blood component includes, without limitation, whole blood (which may be simply referred to as blood), white blood cells including at least about 10% volume plasma, red blood cells including at least about 10% volume plasma, platelets including at least about 10% volume plasma, plasma, and various fractions of blood including at least about 10% volume plasma including the platelet fraction, the red blood cell fraction (e.g., comprised of a majority of red blood cells, and a minority of some white blood cells and plasma), and the buffy coat fraction (e.g., comprised of a majority of white blood cells and platelets, and a minority of some red blood cells and plasma). A blood component also includes any of the above-listed components that also includes a substance (e.g., citric acid or citrate, or heparin) added after the blood component is obtained from the patient that prevents or reduces the coagulation of the blood component.

“Anticoagulant” means a substance (i.e., a reagent or a drug) that prevents or reduces coagulation (i.e., clotting) that is present in a blood component of the patient if that substance is taken by or administered to the patient prior to obtaining the blood component from the patient. Such administration may be by any route including oral, parenteral, intravenous, intraperitoneal, intramuscular, subcutaneous, etc. Note that a substance (e.g., heparin or citrate) that is added to a blood component after the blood component is obtained from the patient is not an anticoagulant within this definition.

“Viscoelastic analysis” means any analysis method that measures the characteristics of elastic solid (e.g., fibrin solids) and fluids. In other words, viscoelastic analysis allows the study of properties of a viscous fluid, such as blood, plasma, or a blood sample. Viscoelastic analysis includes a clotting assay.

A “clotting measurement” is a measurement of clot formation. This measurement can be taken any time during the formation of a clot including, without limitation, the time of initial formation of fibrin. The time of initial formation of fibrin is referred to as an “R-time.”

“Clotting assay” means any type of assay that can be used to measure the ability of blood or a blood component to form a clot. Clotting assays include, without limitation, a viscoelastic assay (including a thromboelastography (TEG) assay or a thromboelastometry (TEM) assay), a prothrombin time (PT) assay, an activated partial thromboplastin time (aPTT) assay, and an activated clotting time (ACT) assay.

“Container” means a rigid surface (e.g., a solid surface), a portion of which contacts a portion of a blood component sample placed into the container at any point during the viscoelastic analysis. The portion of the container that contact the portion of blood component sample may also be referred to as the “interior” of the container. Note that the phrase “into the container” does not mean that the container has a bottom surface which is in contact with the portion of the blood sample. Rather, the container can be a ring-shaped structure, where the inside of the ring is the interior of the container, meaning that the inside of the ring is the portion of the ring-shaped container that contacts a portion of the blood component sample. A blood component sample can flow into the container and be held there, for example, by vacuum pressure or surface tension.

“Therapeutically relevant amount” means an amount of an anticoagulant in the blood component being tested that is within the therapeutically effective concentration range for the anticoagulant. The therapeutically relevant amount will differ for each anticoagulant, and is affected by the bioavailability of the anticoagulant and also the half-life of the anticoagulant following ingestion by the patient. For example, dabigatran has a half-life of 12-17 hours which is lengthened in patients with renal dysfunction (Boehringer Ingelheim International G. Pradaxa (dabigatran etexilate) product information). Apixaban and rivaroxaban have shorter half-lives than dabigatran. However, apixaban has also an increased half-life of up to 44% in patients with severe renal impairment compared to healthy volunteers (see Dager et al., Crit. Care Med. 41: e42-46, 2013). The anticoagulant effect of apixaban or rivaroxaban can be expected to persist for at least 10-30 hours after the last dose, i.e. for about two half-lives. Generally, however, the therapeutically relevant amount of an anticoagulant is between about 75 ng/ml to about 500 ng/ml in the blood (or blood component). For example, for apixaban, a therapeutically relevant amount is between about 275 to about 775 ng/ml, or between about 300 to about 650 ng/ml, or between about 400 to about 600 ng/ml, or at about 500 ng/ml in the blood or blood component. For rivaroxaban, a therapeutically relevant amount is between about 40 to about 350 ng/ml, or between about 55 to about 250 ng/ml, or between about 70 to about 150 ng/ml, or at about 89 ng/ml in the blood or blood component. For dabigatran, a therapeutically relevant amount is between about 100 to about 350 ng/ml, or between about 150 to about 300 ng/ml, or between about 175 to about 250 ng/ml, or at about 200 ng/ml in the blood or blood component. A therapeutically relevant amount of warfarin provides an international normalized ratio (INR) of about 1 to about 4. INR is a measure of how long it takes blood to clot. Therapeutic heparin is provided to patients in one of two forms: unfractionated heparin and low molecular weight heparin (LMWH). A therapeutically relevant amount of unfractionated heparin is about 0.3 to about 0.7 IU/ml. A therapeutically relevant amount of the LMWH enoxaparin sodium is about 0.6 to about 1.0 IU/ml. A therapeutically relevant amount of the LMWH dalteparin sodium is about 0.5 to about 1.05 IU/ml.

“Ecarin reagent” means a molecule that activates a prothrombin zymogen (precursor of active thrombin) and produces an activated form with thrombin-like enzymatic activity. Non-limiting examples of ecarin reagents include ecarin, Taipan venom (derived from the venom of the saw-scaled viper, Echis carinatus), and textarin.

In various embodiments, the methods described herein involve the use of a Factor Xa reagent. “Factor Xa reagent” means Factor Xa (FXa) and/or any combination of clotting Factors that include Factor Xa. This Factor Xa reagent may contain other substances for performance and/or stability improvement (including salts, buffers, sugars etc.). Factor Xa reagent is added to a blood component after that blood component has been obtained from the patient. Alternatively, the Factor Xa reagent may be prepared from the Factor X endogenous in the blood component sample by the addition of another reagent such as Russel's Viper venom that activates the Factor X zymogen (precursor of active Factor Xa).

“Heparinase reagent” means a reagent selected from the group consisting of heparinase, polybrene, and combinations thereof.

As used herein, “neat,” a “neat sample,” and the like, refers to a blood component sample lacking any anticoagulant.

In some embodiments, the invention utilizes a clotting assay to assess the functioning of the clotting cascade in a blood component from a patient.

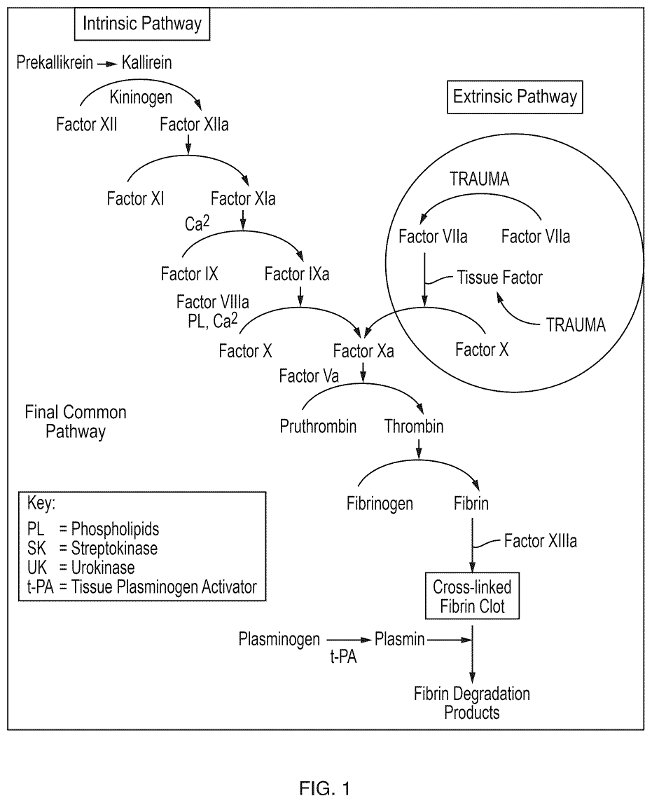

The clotting cascade (or coagulation cascade) is a tightly regulated process by which blood changes from liquid to a solid clot. This process is called coagulation or clotting. FIG. 1 provides a schematic diagram of the clotting cascade. Clotting can be triggered by the extrinsic tissue factor pathway (e.g., by injury or damage to a blood vessel) or by the intrinsic contact activation pathway. The two pathways join in the activation of Factor Xa which then activates prothrombin to thrombin.

Using the devices and methods described herein, the identification and classification of an anticoagulant (if the patient has taken the anticoagulant) and reversal (if a reversal agent has been administered to the patient) can be determined. In some embodiments, the patient is undergoing (or will shortly be undergoing) a condition that may involve bleeding. For example, the patient may be undergoing surgery, may be being prepared for surgery, may be injured or wounded, may be bleeding, or may have had or is currently having or is suspected to imminently have a thromboembolic event including, without limitation, a stroke, a venous thromboembolic event (VTE), a heart attack, heart failure, an arterial thromboembolic event, and a pulmonary embolism. For patients with ischemic stroke, the presence of a relevant anticoagulant may be prohibitive for lysis therapy. Hemorrhagic stroke patients may benefit from anticoagulant reversal. The patient may be a trauma patient and/or may have internal bleeding, where the effects of an anticoagulant may contribute to clinical presentation and potential treatment decisions.

In some embodiments, an anticoagulant is a direct thrombin inhibitor, and may be referred to as a DTI. Thrombin (Clotting Factor Ila) is a central player in the blood clotting process (see FIG. 1). Thrombin plays multiple roles including (a) converting soluble fibrinogen to fibrin; (b) activating factors VI, VIII, XI, and XIII and (c) stimulating platelets. By activating Factors XI and XIII, thrombin generates more thrombin and favors formation of cross-linked fibrin molecules, thereby strengthening the blood clot.

A DTI is an anticoagulant that binds thrombin and blocks thrombin's interaction with its substrates. DTIs may be bivalent (blocking thrombin at the active site and one of the exosites) or univalent (blocking thrombin at the active site). Bivalent DTIs include, without limitation, hirudin and bivalirudin. Univalent DTIs include, without limitation, argatroban, melagatran, ximelagatran, and dabigatran. Dabigatran is sold commercially by Boehringer Ingelheim International GmbH, Ingelheim, Germany under the name Pradaxa. Dabigatran is an oral direct inhibitor of thrombin (Factor IIa) that is “not permanent,” selective and competitive. Dabigatran is licensed in Europe and the USA to reduce the risk of venous thromboembolism (VTE) in orthopedic surgical patients as well as stroke and systemic embolism in patients with non-valvular atrial fibrillation.

In some embodiments, an anticoagulant is an inhibitor of Factor Xa and may be referred to as an anti-Factor Xa reagent, a Factor Xa inhibitor, or an xaban. An xaban acts directly upon Factor Xa in the blood clotting cascade (see FIG. 1). Two non-limiting commercially available inhibitors of Factor Xa are Rivaroxaban (sold under the name of Xarelto by Bayer Pharma AG, Leverkusen, Germany and Janssen Pharmaceuticals, Inc., Titusville, New Jersey) and Apixaban (sold under the name Eliquis by Bristol-Myers Squibb, New York, New York and Pfizer EEIG Sandwich, United Kingdom). Rivaroxaban and Apixaban are licensed in Europe and the USA to reduce the risk of venous thromboembolism (VTE) in orthopedic surgical patients as well as stroke and systemic embolism in patients with non-valvular atrial fibrillation. Rivaroxaban is also approved in the EU for the secondary prevention of acute coronary syndrome. Rivaroxaban can be administered in combination with acetylsalicylic acid (ASA) or with ASA plus clopidogrel or ticlopidine for the prevention of thrombotic events in adult patients with elevated cardiac biomarkers after a coronary event according to the product information provided by Bayer Pharma.

Additional non-limiting examples of Factor Xa inhibitors include betrixaban (LY517717; Portola Pharmaceuticals), darexaban (YM150; Astellas), edoxaban (Lixiana; DU-176b; Daiichi), TAK-442 (Takeda), and eribaxaban (PD0348292; Pfizer).

In some embodiments, an anticoagulant is a vitamin K antagonist. A vitamin K antagonist interferes with vitamin K, disrupting the formation of factors II, VII, IX, and X, as well as proteins C and S. A non-limiting example of a vitamin K antagonist includes warfarin.

In some embodiments, an anticoagulant is heparin. Heparin binds to and activates the enzyme inhibitor antithrombin III (AT). Activated AT then inactivates thrombin, Factor Xa, and other proteases. Non-limiting examples of heparin include unfractionated heparin, enoxaparin sodium, and dalteparin sodium.

In some embodiments, the viscoelastic analysis is performed under conditions that mimic the conditions in vivo that result in hemostasis. For example, the condition may include a temperature that mimics a body temperature (e.g., a temperature of 37° C.). The condition may also include clot formation and dissolution at flow rates that mimic those found in blood vessels.

In some embodiments, viscoelastic analysis of a blood component sample may include subjecting the blood component sample to analysis on a hemostasis analyzer instrument. One non-limiting viscoelastic analysis method is the thromboelastography (“TEG”) assay. Thus in some embodiments, the viscoelastic analysis includes subjecting a blood component sample to analysis using thromboelastography (TEG), which was first described by Helmut Hartert in Germany in the 1940's.

Various devices that perform thromboelastography, and methods for using it are described in U.S. Pat. Nos. 5,223,227; 6,225,126; 6,537,819; 7,182,913; 6,613,573; 6,787,363; 7,179,652; 7,732,213, 8,008,086; 7,754,489; 7,939,329; 8,076,144; 6,797,419; 6,890,299; 7,524,670; 7,811,792; 8,421,458; 7,261,861, 10,954,549 and 10,501,773; and U.S. Publication Nos. 2007/0092405; 2007/0059840; and 2012/0301967; the entire disclosures of each of which are hereby expressly incorporated herein by reference.

Thromboelastography (TE) monitors the elastic properties of a blood component as it is induced to clot under a low shear environment resembling sluggish venous blood flow. The patterns of changes in shear elasticity of the developing clot enable the determination of the kinetics of clot formation, as well as the strength and stability of the formed clot; in short, the mechanical properties of the developing clot. As described above, the kinetics, strength and stability of the clot provides information about the ability of the clot to perform “mechanical work,” i.e., resisting the deforming shear stress of the circulating blood. In essence, the clot is the elementary machine of hemostasis. Hemostasis instruments that measure hemostasis are able to measure the ability of the clot to perform mechanical work throughout its structural development. These hemostasis analyzers measure continuously all phases of patient hemostasis as a net product of whole blood components in a non-isolated, or static fashion from the time of test initiation until initial fibrin formation, through clot rate strengthening and ultimately clot strength through clot lysis.

In some embodiments, the viscoelastic analysis and/or the hemostasis analyzer comprises a container which is in contact with the blood component sample.

Still additional types of containers that are included in this definition are those present on plates and cassettes (e.g., a microfluidic cassette), where the plate or cassette has multiple channels, reservoirs, tunnels, and rings therein. Each of the contiguous channels (comprising, for example, a channel, a reservoir, and a ring) is a container, as the term is used herein. Hence, there may be multiple containers on one cassette. U.S. Pat. No. 7,261,861 (incorporated herein by reference) describes such a cassette with multiple channels or containers. Any of the surfaces in any of the channels or tunnels of the cassette may be an interior of the container if that surface comes into contact with any portion of the blood sample, at any time during the viscoelastic analysis.

One non-limiting hemostasis analyzer instrument is described in U.S. Pat. No. 7,261,861; US Patent Publication No. US US20070092405; and US Patent Publication No. US20070059840.

Another non-limiting hemostasis analyzer instrument that performs viscoelastic analysis using thromboelastography is the TEG thromboelastograph hemostasis analyzer system sold commercially by Haemonetics, Corp. (Braintree, MA).

Thus, the TEG assay may be performed using the TEG thromboelastograph hemostasis analyzer system that measures the mechanical strength of an evolving blood clot. To run the assay, the blood component sample is placed into a container (e.g., a cup or a cuvette), and a plastic pin goes into the center of the container. Contact with the interior walls of the container (or addition of a clot activator to the container) initiates clot formation. The TEG thromboelastograph hemostasis analyzer then rotates the container in an oscillating fashion, approximately 4.45 degrees to 4.75 degrees, every 10 seconds, to imitate sluggish venous flow and activate coagulation. As fibrin and platelet aggregates form, they connect the inside of the container with the plastic pin, transferring the energy used to move the container in the pin. A torsion wire connected to the pin measures the strength of the clot over time, with the magnitude of the output directly proportional to the strength of the clot.

The rotational movement of the pin is converted by a transducer to an electrical signal, which can be monitored by a computer including a processor and a control program. The computer is operable on the electrical signal to create a hemostasis profile corresponding to the measured clotting process. Additionally, the computer may include a visual display or be coupled to a printer to provide a visual representation of the hemostasis profile.

A clotting assay may be used to measure an R-time of a given blood component sample, which is the period of time of latency from the time that a blood component is subjected to viscoelastic analysis until initial fibrin formation. This typically takes about 30 second to about 10 minutes; however the R-time will vary based on the assay performed (e.g., type of blood component being tested, whether the blood component is citrated or not, etc.). For patients in a hypocoagulable state (i.e., a state of decreased coagulability of blood), the R-time is longer/greater than that of a patient in a non-hypocoagulable state, which indicates slower clot formation, while in a hypercoagulable state (i.e., a state of increased coagulability of blood), the R-time is shorter/lower. As described herein, R-time (in minutes or seconds) is non-limiting clotting measurement.

Viscoelastic measurements of coagulation provided by devices such as TEG are increasingly being employed to assess trauma patients who arrive in shock secondary to massive bleeding as well as for acute care of surgical patients with bleeding diatheses. TEG is widely used as a management tool for cardiac surgery and transplant patients and provides information to guide administration of blood products (see Holcolmb J. B. et al., Ann. Surg. 256:476-486, 2012).

Another viscoelastic hemostasis assay that can be used is the thromboelastometry (“TEM”) assay. This TEM assay may be performed using the ROTEM Thromboelastometry Coagulation Analyzer (TEM International GmbH, Munich, Germany), the use of which is well known (See, e.g., Sorensen, B., et al., J. Thromb. Haemost., 2003. 1 (3): p. 551-8. Ingerslev, J., et al., Haemophilia, 2003. 9 (4): p. 348-52. Fenger-Eriksen, C., et al. Br J Anaesth, 2005. 94 (3): p. 324-9]. In the ROTEM analyzer, the blood component sample is placed into a container (also called a cuvette or cup) and a cylindrical pin is immersed. Between pin and the interior wall of the container there is a gap of 1 mm which is bridged by the blood. The pin is rotated by a spring to the right and the left. As long as the blood is liquid (i.e., unclotted), the movement is unrestricted. However, when clotting starts, the clot increasingly restricts the rotation of the pin with rising clot firmness. The pin is connected to an optical detector. This kinetic is detected mechanically and calculated by an integrated computer.

In the ROTEM Thromboelastometry Coagulation Analyzer, the movement of the pin can be monitored by a computer including a processor and a control program. The computer is operable on the electrical signal to create a hemostasis profile corresponding to the measured clotting process.

In some embodiments, the container is a disposable cartridge, e.g., a microfluidic cassette, or a particular channel in the disposable cartridge, the blood component sample may be excited at a resonant frequency and its behavior observed by an electromagnetic or light source as coagulation occurs. In other embodiments the blood component sample's clotting measurement may be observed for changes with a light source without exciting the sample.

Because a single disposable cartridge may have multiple containers (e.g., different channels in the cartridge), the different samples (e.g., portions of the blood component from the patient) are easily directly comparable one another. For example, one channel may be untreated, one channel may be treated with a first reagent, e.g., a Factor Xa reagent and calcium chloride, a different channel may be treated, e.g., with an ecarin reagent, and so on. In another example, blood components from different individuals can be measured in different channels, and the results from the different individuals obtained simultaneously from a single cassette.

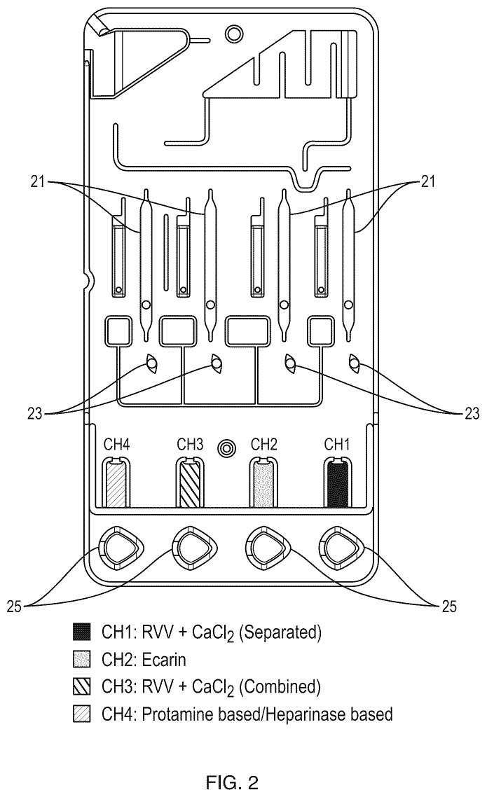

FIG. 2 shows an exemplary disposable cartridge comprising four channels, each channel having a receiving chamber 21 fluidically coupled to a reagent chamber 23, which is fluidically coupled to a measurement chamber 25 (fluidic couplings not shown). For each channel, a sample of a blood component from a patient flows from the receiving chamber to the reagent chamber, and finally to the measurement chamber, where the sample is subject to viscoelastic analysis.

In some embodiments, the invention provides methods and devices for detecting the presence of an anticoagulant in a blood component (e.g., from a subject/patient). In some embodiments, the invention provides methods and devices for classifying the type of anticoagulant is present in the blood component.

In a first aspect, the invention provides a disposable cartridge for detecting and classifying an anticoagulant at a therapeutically relevant amount or higher in a patient, the cartridge comprising a first channel having a first receiving chamber, comprising calcium chloride, configured to receive a first sample of a blood component from the patient, a first reagent chamber, comprising Russel Viper Venom (RVV), fluidically coupled to the first receiving chamber and configured to receive the first sample from the first receiving chamber, and a first measurement chamber fluidically coupled to the first reagent chamber and configured to receive the first sample from the first reagent chamber, the first measurement chamber being further configured for viscoelastic analysis of the first sample received from the first reagent chamber so as to provide a first clotting measurement of the first sample received from the first reagent chamber.

The disposable cartridge also comprises a second channel comprising a second receiving chamber configured to receive a second sample of the blood component from the patient, a second reagent chamber, comprising an ecarin reagent, fluidically coupled to the second receiving chamber and configured to receive the second sample from the second receiving chamber, and a second measurement chamber fluidically coupled to the second reagent chamber and configured to receive the second sample from the second reagent chamber, the second measurement chamber being further configured for viscoelastic analysis of the second sample received from the second reagent chamber so as to provide a second clotting measurement of the second sample received from the second reagent chamber.

In some embodiments, the disposable cartridge further comprises a third channel. The third channel comprises a third receiving chamber configured to receive a third sample of the blood component from the patient, a third reagent chamber, comprising chloride Factor Xa reagent, fluidically coupled to the third receiving chamber and configured to receive the third sample from the third receiving chamber, and a third measurement chamber fluidically coupled to the third reagent chamber and configured to receive the third sample from the third reagent chamber, the third measurement chamber being further configured for viscoelastic analysis of the third sample received from the third reagent chamber so as to provide a third clotting measurement of the third sample received from the third reagent chamber.

In some embodiments the disposable cartridge further comprises a fourth channel, the fourth channel comprising a fourth receiving chamber, comprising protamine, configured to receive a fourth sample of the blood component from the patient, a fourth reagent chamber fluidically coupled to the fourth receiving chamber and configured to receive the fourth sample from the fourth receiving chamber, and a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber. The fourth reagent chamber may comprise a reagent selected from the group consisting of kaolin, tissue factor, calcium chloride, and combinations thereof.

In other embodiments, the disposable cartridge further comprises a fourth channel comprising a fourth receiving chamber, comprising a heparinase reagent, configured to receive a fourth sample of the blood component from the patient, a fourth reagent chamber, comprising kaolin, tissue factor, and calcium chloride, fluidically coupled to the fourth receiving chamber and configured to receive the fourth sample from the fourth receiving chamber, and a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber.

In some embodiments, the first clotting measurement is a first R-time, the second clotting measurement is a second R-time, the third clotting measurement is a third R-time, and/or the fourth clotting measurement is a fourth R-time.

The first channel of the disposable cartridge is configured to detect, at a therapeutically relevant amount or higher, the presence of a Factor Xa inhibitor (such as apixaban, rivaroxaban, and edoxaban), a direct thrombin inhibitor (DTI) (such as dabigatran, argatroban, melagatran, and ximelagatran), and/or heparin in a sample of a blood component from a patient using Russel Viper Venom (RVV) and calcium chloride. RVV is spotted in the reagent chamber of the first channel and calcium chloride is spotted in the receiving chamber of the first channel. Thus, a sample of a blood component from a patient in the first channel will first mix with calcium chloride before mixing with RVV because, after the sample is received by the receiving chamber, the sample flows to the reagent chamber where it is mixed with the RVV spotted therein, before then flowing to the measurement chamber of the first channel. The independent spot locations of calcium chloride and RVV provide the first channel with a greater sensitivity to the presence of a Factor Xa inhibitor in the sample of the blood component from the patient, as measured by changes in reaction time (R-Time), and a relatively lesser sensitivity to the presence of a vitamin K antagonist (VKA) in the sample.

In some embodiments, the second channel of the disposable cartridge is configured to detect, at a therapeutically relevant amount or higher, the presence of a direct thrombin inhibitor (DTI) (such as dabigatran, argatroban, melagatran, and ximelagatran) in a sample of a blood component from a patient using an ecarin reagent, which is spotted in the reagent chamber of the second channel. In other embodiments, the second channel is configured to detect, at a therapeutically relevant amount or higher, the presence of a DTI and heparin in a sample of a blood component from a patient using an ecarin reagent.

The third channel of the disposable cartridge is configured to detect, at a therapeutically relevant amount or higher, the presence of a Factor Xa inhibitor (such as apixaban, rivaroxaban, and edoxaban), a direct thrombin inhibitor (DTI) (such as dabigatran, argatroban, melagatran, and ximelagatran), heparin, and/or a vitamin K antagonist (such as warfarin) in a sample of a blood component from a patient using a Factor Xa reagent and calcium chloride. The Factor Xa reagent and calcium chloride are each spotted in the reagent well of the third channel. Thus, as the sample flows from the receiving chamber to the reagent chamber, the sample is mixed with the Factor Xa reagent and calcium chloride concurrently before flowing to the measurement chamber of the third channel. Spotting the Factor Xa reagent and calcium chloride together in the reagent chamber of the third channel yields less sensitivity to the presence of a Factor Xa inhibitor, and a greater sensitivity to the presence of a vitamin K antagonist, compared to the first channel.

In another aspect, the invention provides a method for detecting a presence of and classifying an anticoagulant at a therapeutically relevant amount or higher in a patient. If a specified anticoagulant is not detected as being present at a therapeutically relevant amount or higher, the specified anticoagulant is determined to be present at less than a therapeutically relevant amount, or absent altogether. The method comprises (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, and (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently.

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, and (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher; wherein (a) the first R-time greater than the first control R-time, the second R-time less than or equal to the second control R-time, and the third R-time greater than the third control R-time indicates the presence of the Factor Xa inhibitor at or above a therapeutically relevant amount in the patient; (b) the first R-time greater than the first control R-time, the second R-time greater than the second control R-time, and the third R-time greater than the third control R-time indicates the presence of the DTI at or above a therapeutically relevant amount in the patient; and (c) the first R-time less than or equal to the first control R-time, the second R-time less than or equal to the second control R-time, and the third R-time greater than the third control R-time indicates the presence of the vitamin K antagonist at or above a therapeutically relevant amount in the patient.

In another aspect, the invention provides a method for detecting a presence of and classifying an anticoagulant at a therapeutically relevant amount or higher in a patient, the method comprising (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and (D) determining a fourth R-time by subjecting a fourth sample of the blood component from the patient to a clotting assay in the presence of protamine.

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher, and (iv) comparing the fourth R-time to a fourth control R-time, said fourth control R-time being derived from a fourth set of control blood component samples, each control blood component sample of the fourth set having been obtained from a control patient known to lack each of a Factor Xa inhibitor, a DTI, a vitamin K antagonist, and heparin, at a therapeutically relevant amount or higher; wherein (a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient; (b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient; (c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient; (d) the first R-time greater than the first control R-time, the second R-time less than or equal to the second control R-time, the third R-time greater than the third control R-time, and the fourth R-time less than the fourth control R-time indicates the presence of heparin at or above a therapeutically relevant amount in the patient. In some embodiments, the fourth sample of the blood component from the patient may subjected to the clotting assay in the presence of the protamine and an additional reagent selected from the group consisting of kaolin, tissue factor, calcium chloride, and combinations thereof, said fourth sample of the blood component from the patient being admixed with the protamine prior to being admixed with the additional reagent.

In another aspect, the invention provides a method for detecting a presence of and classifying an anticoagulant at a therapeutically relevant amount or higher in a patient, the method comprising (A) determining a first R-time by subjecting a first sample of a blood component from the patient to a first clotting assay in the presence of RVV and calcium chloride, said first sample being admixed with the calcium chloride prior to being admixed with the RVV, (B) determining a second R-time by subjecting a second sample of the blood component from the patient to a second clotting assay in the presence of an ecarin reagent, (C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and (D) determining a fourth R-time by subjecting a fourth sample of the blood component from the patient to a clotting assay in the presence of a heparinase reagent, kaolin, tissue factor, and calcium chloride, said fourth sample of the blood component from the patient being admixed with the heparinase reagent prior to being admixed with the kaolin, tissue factor, and calcium chloride; and

The method further comprises (i) comparing the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher, (ii) comparing the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, (iii) comparing the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher; (iv) comparing the fourth R-time to a fourth control R-time, said fourth control R-time being derived from a fourth set of control blood component samples, each control blood component sample of the fourth set having been obtained from a control patient known to have a control anticoagulant selected from the group consisting of a Factor Xa inhibitor, a DTI, a vitamin K antagonist, and combinations thereof, at a therapeutically relevant amount or higher, and (v) comparing the second R-time to a fifth control R-time, said fifth control R-time being derived from a fifth set of control blood component samples, each control blood component sample of the fifth set having been obtained from a control patient known to lack heparin at a therapeutically relevant amount or higher; wherein (a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient; (b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient; (c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient; (d) the first R-time greater than the first control R-time, the second R-time greater than the fifth control R-time, the third R-time greater than the third control R-time, and the fourth R-time less than the fourth control R-time indicates the presence of heparin at or above a therapeutically relevant amount in the patient.

The Factor Xa inhibitor may be rivaroxaban, edoxaban, or apixaban.

The DTI may be argatroban, melagatran, ximelagatran, or dabigatran.

The vitamin K antagonist may be warfarin.

In some embodiments, the patient is administered a reversal agent so as to reduce the anticoagulant effect of the anticoagulant detected to be present at the therapeutically relevant amount or above in the patient.

In some embodiments, a control R-time may be an average, a median, or a range of at least two R-time measurements of at least two control blood component samples, each control blood component sample having been obtained from a control patient known to lack at least one anticoagulant at a therapeutically relevant amount or higher, in accordance with the requirements of a specific assay (such as the first control R-time, the second control R-time, the third control R-time, a fourth control R-time, and the fifth control R-time described herein). For example, a range (which may be referred to as a reference range) may be created from multiple control blood component samples (e.g., from multiple donors known to lack at least one anticoagulant at a therapeutically relevant amount or higher, in accordance with the requirements a specific assay). If an average or mean is used, the R-time measurements from multiple control blood component samples are averaged, and that average number is used as a given control R-time measurement. A typical such control R-time will be 1-4 minutes.

In some embodiments, a control R-time may be an average, a median, or a range of at least two R-time measurements of at least two control blood component samples, each control blood component sample having been obtained from a control patient known to have at least one anticoagulant at a therapeutically relevant amount or higher, in accordance with the requirements of a specific assay (such as a fourth control R-time described herein). For example, a range (which may be referred to as a reference range) may be created from multiple control blood component samples (e.g., from multiple donors known to have at least one anticoagulant at a therapeutically relevant amount or higher, in accordance with the requirements a specific assay). If an average or mean is used, the R-time measurements from multiple control blood component samples are averaged, and that average number is used as a given control R-time measurement. A typical such control R-time will be above 4 minutes.

Table 1 provides an exemplary outcome table for detecting and classifying an anticoagulant at a therapeutically relevant amount or higher in a patient using a four channel cartridge comprising protamine in the fourth channel.

TABLE 1
Outcomes for an Anticoagulant Present at a Therapeutically Relevant Amount
or Higher for a Cartridge comprising Protamine in the Fourth Channel

| Anticoagulant | First Channel | Second | Third Channel | Fourth |
|---|---|---|---|---|
| Present | Assay | Channel Assay | Assay | Channel Assay |
| Factor Xa | First R-time | Second R-time | Third R-time | Fourth R-time |
| Inhibitor | greater than | less than or | greater than | greater than or |
| | first control | equal to second | third control | equal to fourth |
| | R-time | control R-time | R-time | control R-time |
| DTI | First R-time | Second R-time | Third R-time | Fourth R-time |
| | greater than | greater than | greater than | greater than or |
| | first control | second control | third control | equal to fourth |
| | R-time | R-time | R-time | control R-time |
| VKA | First R-time | Second R-time | Third R-time | Fourth R-time |
| | less than or | less than or | greater than | greater than or |
| | equal to first | equal to second | third control | equal to fourth |
| | control R-time | control R-time | R-time | control R-time |
| Heparin | First R-time | Second R-time | Third R-time | Fourth R-time |
| | greater than | less than or | greater than | less than fourth |
| | first control | equal to second | third control | control R-time |
| | R-time | control R-time | R-time | |
| None | First R-time | Second R-time | Third R-time | Fourth R-time |
| | less than or | less than or | less than or | greater than or |
| | equal to first | equal to second | equal to third | equal to fourth |
| | control R-time | control R-time | control R-time | control R-time |

Table 2 provides an exemplary outcome table for detecting and classifying an anticoagulant at a therapeutically relevant amount or higher in a patient using a four channel cartridge comprising a heparinase reagent in the fourth channel.

TABLE 2
Outcomes for an Anticoagulant Present at a Therapeutically Relevant Amount or
Higher for a Cartridge comprising a Heparinase Reagent in the Fourth Channel

| Anticoagulant | First Channel | Second | Third Channel | Fourth |
|---|---|---|---|---|
| Present | Assay | Channel Assay | Assay | Channel Assay |
| Factor Xa | First R-time | Second R-time | Third R-time | Fourth R-time |
| Inhibitor | greater than | less than or | greater than | greater than or |
| | first control | equal to second | third control | equal to fourth |
| | R-time | control R-time | R-time | control R-time |
| DTI | First R-time | Second R-time | Third R-time | Fourth R-time |
| | greater than | greater than | greater than | greater than or |
| | first control | second control | third control | equal to fourth |
| | R-time | R-time | R-time | control R-time |
| VKA | First R-time | Second R-time | Third R-time | Fourth R-time |
| | less than or | less than or | greater than | greater than or |
| | equal to first | equal to second | third control | equal to fourth |
| | control R-time | control R-time | R-time | control R-time |
| Heparin | First R-time | Second R-time | Third R-time | Fourth R-time |
| | greater than | greater than | greater than | less than fourth |
| | first control | fifth control | third control | control R-time |
| | R-time | R-time | R-time | |
| None | First R-time | Second R-time | Third R-time | Fourth R-time |
| | less than or | less than or | less than or | less than or |
| | equal to first | equal to second | equal to third | equal to fourth |
| | control R-time | control R-time | control R-time | control R-time |

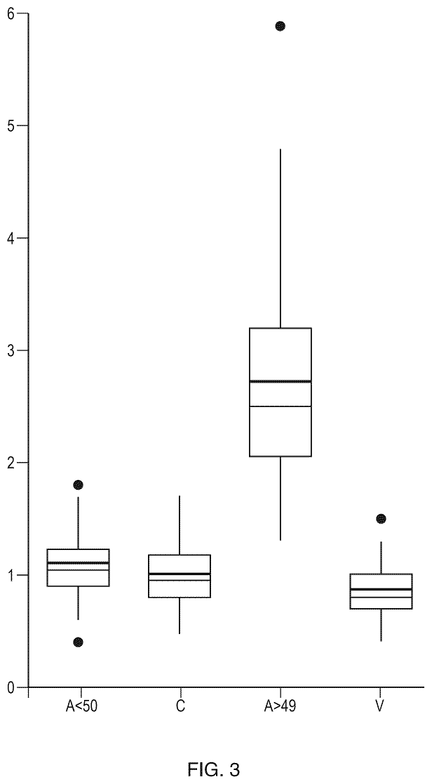

FIG. 3 shows R-time (Y-axis) results obtained using the first channel of a cartridge described herein (calcium chloride spotted in receiving chamber, RVV spotted in reagent chamber). Blood component samples from 42 control patients lacking any anticoagulant at a therapeutically relevant amount or higher (“C”), blood component samples from 38 patients having a therapeutically relevant amount of a vitamin K antagonist (“V”) (with INR values of 1.67-3.84), blood component samples from 20 patients having apixaban concentrations <50 ng/ml (“A<50”), and blood component samples from 55 patients having apixaban concentrations >=50 ng (“A>49”). These results demonstrate a relative lack of sensitivity to a vitamin K antagonist compared to a Factor Xa inhibitor for the first channel.

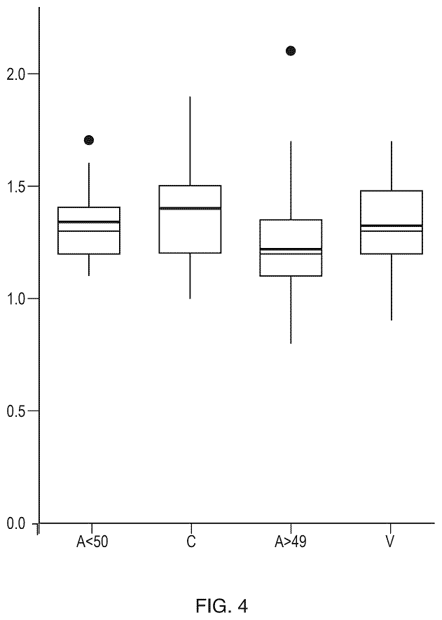

FIG. 4 shows R-time results (Y-axis) obtained using the second channel of a cartridge described herein (ecarin spotted in reagent chamber). Blood component samples from 42 control patients lacking any anticoagulant at a therapeutically relevant amount or higher (“C”), blood component samples from 38 patients having a therapeutically relevant amount of a vitamin K antagonist (“V”) (with INR values of 1.67-3.84), blood component samples from 20 patients having apixaban concentrations <50 ng/ml (“A<50”), and blood component samples from 55 patients having apixaban concentrations >=50 ng (“A>49”) were assayed.

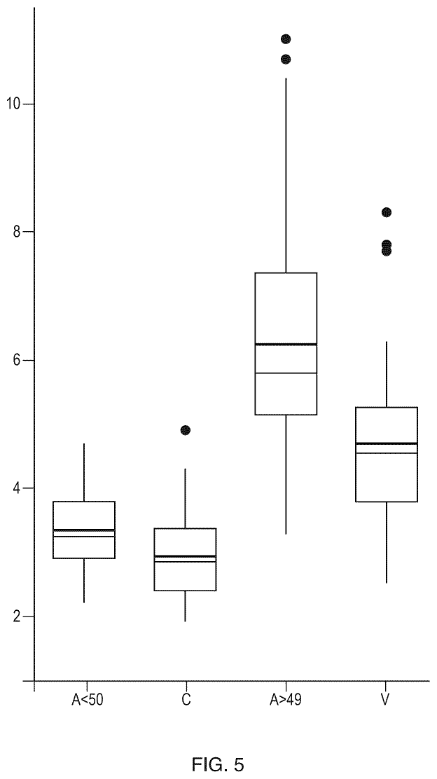

FIG. 5 shows R-time results (Y-axis) obtained using the third channel of a cartridge described herein (calcium chloride and RVV spotted in reagent chamber). Blood component samples from 42 control patients lacking any anticoagulant at a therapeutically relevant amount or higher (“C”), blood component samples from 38 patients having a therapeutically relevant amount of a vitamin K antagonist (“V”) (with INR values of 1.67-3.84), blood component samples from 20 patients having apixaban concentrations <50 ng/ml (“A<50”), and blood component samples from 55 patients having apixaban concentrations >=50 ng (“A>49”) were assayed. These results demonstrate high sensitivity to a vitamin K antagonist.

In some embodiments, once an anticoagulant has been detected and classified at a therapeutically relevant amount or higher in a patient, if desired, the patient can be treated with a reversal agent (e.g., in a therapeutically relevant amount). For example, if the patient is identified as having a dabigatran anticoagulant (a DTI anticoagulant), a non-limiting reversal agent that can be administered to the patient to reverse the anticoagulant effect of the dabigatran is Idarucizumab (Boehringer Ingelheim). Similarly, if the patient is identified as having a Factor Xa inhibitor anticoagulant, a non-limiting reversal agent that can be administered to the patient to reverse the anticoagulant effect of the Factor Xa inhibitor is andexanet alfa (Portola Pharmaceuticals). Another non-limiting reversal agent that can be administered to the patient to reverse the anticoagulant effect of a DTI or a Factor Xa inhibitor anticoagulant is prothrombin complex concentrates (PCC) (for example, the PCC-4 factor sold under the name KCentra, Octaplex, and Beriplex).

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A disposable cartridge for detecting and classifying an anticoagulant at a therapeutically relevant amount or higher in a patient, the cartridge comprising:
(1) a first channel having:
(a) a first receiving chamber, comprising calcium chloride, configured to receive a first sample of a blood component from the patient,
(b) a first reagent chamber, comprising Russel Viper Venom (RVV), fluidically coupled to the first receiving chamber and configured to receive the first sample from the first receiving chamber, and
(c) a first measurement chamber fluidically coupled to the first reagent chamber and configured to receive the first sample from the first reagent chamber, the first measurement chamber being further configured for viscoelastic analysis of the first sample received from the first reagent chamber so as to provide a first clotting measurement of the first sample received from the first reagent chamber; and
(2) a second channel having:
(d) a second receiving chamber configured to receive a second sample of the blood component from the patient,
(e) a second reagent chamber, comprising an ecarin reagent, fluidically coupled to the second receiving chamber and configured to receive the second sample from the second receiving chamber, and
(f) a second measurement chamber fluidically coupled to the second reagent chamber and configured to receive the second sample from the second reagent chamber, the second measurement chamber being further configured for viscoelastic analysis of the second sample received from the second reagent chamber so as to provide a second clotting measurement of the second sample received from the second reagent chamber.

2. The disposable cartridge of claim 1, the cartridge further comprising:
(3) a third channel having:
(g) a third receiving chamber configured to receive a third sample of the blood component from the patient,
(h) a third reagent chamber, comprising a Factor Xa reagent and calcium chloride, fluidically coupled to the third receiving chamber and configured to receive the third sample from the third receiving chamber, and
(i) a third measurement chamber fluidically coupled to the third reagent chamber and configured to receive the third sample from the third reagent chamber, the third measurement chamber being further configured for viscoelastic analysis of the third sample received from the third reagent chamber so as to provide a third clotting measurement of the third sample received from the third reagent chamber.

3. The disposable cartridge of claim 2, the cartridge further comprising:
(4) a fourth channel having
(j) a fourth receiving chamber, comprising protamine, configured to receive a fourth sample of the blood component from the patient,
(k) a fourth reagent chamber fluidically coupled to the fourth receiving chamber and configured to receive the fourth sample from the fourth receiving chamber, and
(l) a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber.

4. The disposable cartridge of claim 2, the cartridge further comprising:
(4) a fourth channel having
(j) a fourth receiving chamber, comprising a heparinase reagent, configured to receive a fourth sample of the blood component from the patient,
(k) a fourth reagent chamber, comprising kaolin, tissue factor, and calcium chloride, fluidically coupled to the fourth receiving chamber and configured to receive the fourth sample from the fourth receiving chamber, and
(l) a fourth measurement chamber fluidically coupled to the fourth reagent chamber and configured to receive the fourth sample from the fourth reagent chamber, the fourth measurement chamber being further configured for viscoelastic analysis of the fourth sample received from the fourth reagent chamber so as to provide a fourth clotting measurement of the fourth sample received from the fourth reagent chamber.

5. The disposable cartridge of claim 3, wherein the fourth reagent chamber comprises a reagent selected from the group consisting of kaolin, tissue factor, calcium chloride, and combinations thereof.

6. The disposable cartridge according to claim 1, wherein the first clotting measurement is a first R-time.

7. The disposable cartridge according to claim 6, wherein the second clotting measurement is a second R-time.

8. The disposable cartridge according to claim 7, wherein the third clotting measurement is a third R-time.

9. The disposable cartridge according to claim 8, wherein the fourth clotting measurement is a fourth R-time.

10. A method for detecting a presence of and classifying an anticoagulant at a therapeutically relevant amount or higher in a patient, the method comprising:
(A) determining a first R-time by subjecting a first sample of a blood component from the patient to a first clotting assay in the presence of RVV and calcium chloride, said first sample being admixed with the calcium chloride prior to being admixed with the RVV,
(B) determining a second R-time by subjecting a second sample of the blood component from the patient to a second clotting assay in the presence of an ecarin reagent, and
(C) determining a third R-time by subjecting a third sample of the blood component from the patient to a third clotting assay in the presence of a Factor Xa reagent and calcium chloride, said third sample being admixed with the Factor Xa reagent and the calcium chloride concurrently; and
comparing:
(i) the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher,
(ii) the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher, and
(iii) the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher;
wherein:
(a) the first R-time greater than the first control R-time, the second R-time less than or equal to the second control R-time, and the third R-time greater than the third control R-time indicates the presence of the Factor Xa inhibitor at or above a therapeutically relevant amount in the patient;
(b) the first R-time greater than the first control R-time, the second R-time greater than the second control R-time, and the third R-time greater than the third control R-time indicates the presence of the DTI at or above a therapeutically relevant amount in the patient;
(c) the first R-time less than or equal to the first control R-time, the second R-time less than or equal to the second control R-time, the third R-time greater than the third control R-time indicates the presence of the vitamin K antagonist at or above a therapeutically relevant amount in the patient.

11. A method for detecting a presence of and classifying an anticoagulant at a therapeutically relevant amount or higher in a patient, the method comprising:
(A) determining a first R-time by subjecting a first sample of a blood component from the patient to a first clotting assay in the presence of RVV and calcium chloride, said first sample being admixed with the calcium chloride prior to being admixed with the RVV,
(B) determining a second R-time by subjecting a second sample of the blood component from the patient to a second clotting assay in the presence of an ecarin reagent,
(C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and
(D) determining a fourth R-time by subjecting a fourth sample of the blood component from the patient to a clotting assay in the presence of protamine; and
comparing:
(i) the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher,
(ii) the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher,
(iii) the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher, and
(iv) the fourth R-time to a fourth control R-time, said fourth control R-time being derived from a fourth set of control blood component samples, each control blood component sample of the fourth set having been obtained from a control patient known to lack each of a Factor Xa inhibitor, a DTI, a vitamin K antagonist, and heparin, at a therapeutically relevant amount or higher;
wherein:
(a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient;
(b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient;
(c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient;
(d) the first R-time greater than the first control R-time, the second R-time less than or equal to the second control R-time, the third R-time greater than the third control R-time, and the fourth R-time less than the fourth control R-time indicates the presence of heparin at or above a therapeutically relevant amount in the patient.

12. A method for detecting a presence of and classifying an anticoagulant at a therapeutically relevant amount or higher in a patient, the method comprising:
(A) determining a first R-time by subjecting a first sample of a blood component from the patient to a first clotting assay in the presence of RVV and calcium chloride, said first sample being admixed with the calcium chloride prior to being admixed with the RVV,
(B) determining a second R-time by subjecting a second sample of the blood component from the patient to a second clotting assay in the presence of an ecarin reagent,
(C) determining a third R-time by subjecting a third sample of the blood component from the patient to a third clotting assay in the presence of a Factor Xa reagent and calcium chloride, said third sample being admixed with the Factor Xa reagent and the calcium chloride concurrently, and
(D) determining a fourth R-time by subjecting a fourth sample of the blood component from the patient to a clotting assay in the presence of a heparinase reagent, kaolin, tissue factor, and calcium chloride, said fourth sample of the blood component from the patient being admixed with the heparinase reagent prior to being admixed with the kaolin, tissue factor, and calcium chloride; and
comparing:
(i) the first R-time to a first control R-time, said first control R-time being derived from a first set of control blood component samples, each control blood component sample of the first set having been obtained from a control patient known to lack each of a Factor Xa inhibitor, a DTI, and heparin at a therapeutically relevant amount or higher,
(ii) the second R-time to a second control R-time, said second control R-time being derived from a second set of control blood component samples, each control blood component sample of the second set having been obtained from a control patient known to lack a direct thrombin inhibitor (DTI) at a therapeutically relevant amount or higher,
(iii) the third R-time to a third control R-time, said third control R-time being derived from a third set of control blood component samples, each control blood component sample of the third set having been obtained from a control patient known to lack each of a Factor Xa inhibitor, a DTI, heparin, and a vitamin K antagonist at a therapeutically relevant amount or higher;
(iv) the fourth R-time to a fourth control R-time, said fourth control R-time being derived from a fourth set of control blood component samples, each control blood component sample of the fourth set having been obtained from a control patient known to have a control anticoagulant selected from the group consisting of a Factor Xa inhibitor, a DTI, a vitamin K antagonist, and combinations thereof, at a therapeutically relevant amount or higher, and
(v) the second R-time to a fifth control R-time, said fifth control R-time being derived from a fifth set of control blood component samples, each control blood component sample of the fifth set having been obtained from a control patient known to lack heparin at a therapeutically relevant amount or higher;
wherein:
(a) the first R-time greater than the first control R-time, the second R-time less than or equal to the second control R-time, the third R-time greater than the third control R-time, and the fourth R-time greater than or equal to the fourth control R-time indicates the presence of the Factor Xa inhibitor at or above a therapeutically relevant amount in the patient;
(b) the first R-time greater than the first control R-time, the second R-time greater than the second control R-time, the third R-time greater than the third control R-time, and the fourth R-time greater than or equal to the fourth control R-time indicates the presence of the DTI at or above a therapeutically relevant amount in the patient;
(c) the first R-time less than or equal to the first control R-time, the second R-time less than or equal to the second control R-time, the third R-time greater than the third control R-time, and the fourth R-time greater than or equal to the fourth control R-time indicates the presence of the vitamin K antagonist at or above a therapeutically relevant amount in the patient;
(d) the first R-time greater than the first control R-time, the second R-time greater than the fifth control R-time, the third R-time greater than the third control R-time, and the fourth R-time less than the fourth control R-time indicates the presence of heparin at or above a therapeutically relevant amount in the patient.

13. The method of claim 11, wherein for step (D), the fourth sample of the blood component from the patient is subjected to the clotting assay in the presence of the protamine and an additional reagent selected from the group consisting of kaolin, tissue factor, calcium chloride, and combinations thereof, said fourth sample of the blood component from the patient being admixed with the protamine prior to being admixed with the additional reagent.

14. The method according to claim 12, wherein the Factor Xa inhibitor is selected from the group consisting of rivaroxaban, edoxaban, and apixaban.

15. The method according to claim 12, wherein the DTI is selected from the group consisting of argatroban, melagatran, ximelagatran, and dabigatran.

16. The method according to claim 12, wherein the vitamin K antagonist is warfarin.

17. The method according to claim 12, wherein the patient is administered a reversal agent so as to reduce the anticoagulant effect of the anticoagulant detected to be present at the therapeutically relevant amount or above in the patient.
